(19) United States
(12) Patent Application Publication
(10) Pub. No.: US 2012/0265158 A1
(43) Pub. Date: Oct. 18, 2012

(54) Transdermal Patch Formulation

(75) Inventors: Sebastian Braun, Wermelskirchen (DE); Armin Breitenbach, Leverkusen (DE); Ulrich Becker, Langenfeld (DE)

(73) Assignee: LABTEC GMBH, Langenfeld (DE)

(21) Appl. No.: 13/394,204

(22) PCT Filed: Sep. 9, 2010
(86) PCT No.: PCT/EP10/05546

§ 371 Date: Jun. 20, 2012

(30) Foreign Application Priority Data
Sep. 9, 2009 (EP) 09 011533.8

(51) Int. Cl. A61K 31/135; A61P 25/16; A61M 35/00; B05D 5/00; B05D 5/10; A61K 9/70; A61P 25/24

(52) U.S. Cl. 604/307; 424/443; 514/657; 427/2.31

(57) ABSTRACT

A method for preparing a transdermal patch comprising a substrate layer is provided, the method comprising the steps of contacting an active pharmaceutical ingredient with a retaining means to provide a composition, applying the composition obtained in step (a) to a carrier material to form a substrate layer of the transdermal patch, wherein the retaining means remain within the final transdermal patch.

The invention relates to the field of pharmaceutical patch formations, in particular patch formulations containing rasagiline or derivatives or analogues thereof.

Rasagiline, a selective inhibitor of monoamine oxidase-B, is known for the treatment of central nervous system diseases such as Parkinson's disease (PD), depression, etc., and has gone on sale in Europe. The chemical structure of rasagiline is as follows:

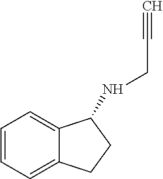

Rasagiline is the active ingredient in the anti Parkinson medicament market in Europe under the brand name Azilect®. This is a tablet which contains a salt of rasagiline, namely the rasagiline mesylate. The tablet has to be taken once daily.

Since the majority of Parkinson patients have difficulties in walking and oral taking, it is desirable to formulate rasagiline in a transdermal administration so that rasagiline may be administered less frequently than every other day or more. Desirable is an administration which is for example only twice or once a week.

The administration of an active ingredient (active pharmaceutical ingredient) as a patch formulation requires that the active ingredient can sufficiently penetrate the skin. In case of rasagiline this is thought to require the use of the free base, since the solubility of the salt thereof in a lipophilic polymer matrix used in transdermal patch formulation as well as its penetration into the skin is too little.

WO2007/101400 A1 describes a transdermal patch formulation containing rasagiline salt. In order to overcome the drawbacks thereof in terms of limited solubility and penetration rate into the skin, this patent application suggests to further add a penetration regulator and a penetration enhancer into the patch formulation. If rasagiline free base is contained in the formulation of WO2007/101400 A1 it is in situ converted from the rasagiline salt into the free base by the addition of bases. However this conversion is disadvantageous since it requires additional steps in the manufacture process and furthermore raises unwanted regulatory hurdles since the conversion may be incomplete.

The patch formulation of WO2007/101400 A1 can either be manufactured according to the standard hot melt technique or according to the standard solvent/casting/drying process. In the hot melt technique, the active ingredient is added to a melted polymer material at a temperature of 50 to 200° C. The molten material is then applied to an inert backing layer and cooled to form the substrate layer. In the solvent/casting/drying technique, the active ingredient is dispersed in a volatile solvent first, and then an organic polymer material is added to the solution. This mixture then is used to form the substrate layer, whereby the solvent is evaporated. The drying step may be performed after the substrate layer has been formed and usually involves temperatures of 50 to 120° C. Such elevated temperatures are considered to be essential for completing the drying procedure at all or at least quickly enough to allow an economically reasonable manufacturing process.

The inventors now found, that applying these techniques to the manufacture of a patch formulation containing the free base of rasagiline inevitably leads to a substantial loss of the active ingredient, which can even be up to 90 or 95% of the originally introduced amount of the active ingredient. This is most likely due to the volatility of the active ingredient causing the active ingredient to sublime or to evaporate during the manufacturing process or to be carried along with evaporating solvent during a drying step. Rasagiline base has a low melting point of 42° C. and is known to sublimate already at low temperatures (e.g. lower than 42° C.). Consequently, the manufacture of transdermal patch formulations comprising rasagiline base according to the standard hot melt technique or according to the standard solvent/casting/drying process is not possible in a reasonable way.

Thus, it is objective of the present invention to provide novel means for the manufacture of transdermal patch formulations, in particular patch formulations containing an active ingredient/drug substance with a high volatility, in particular the free base of rasagiline, which allows for a reduced loss of active ingredient. In particular this manufacture process should be simple and easy to perform, quick, precise, non-hazardous and/or economical. It is furthermore desired that the resulting transdermal patch is comfortable for a patient to apply and/or to wear, safe, allows an extended wearing time, an effective skin permeation of the active pharmaceutical ingredient and/or an extended period in which the administered amount of active pharmaceutical ingredient increases linearly with wearing time.

This objective is solved by the method according to claim 1. Further embodiments of the invention are subject matter of additional independent or dependent claims. Furthermore novel and favourable patch formulations are suggested.

The core concept of the invention is based on the findings that an incubation of the volatile active ingredient in retaining means before admixing the active ingredient into the polymer matrix (“carrier material”) of the patch formulation or—generally spoken—before applying the active ingredient to the polymer matrix of the patch formulation results in a substantially reduced loss of active ingredient during the manufacture process. Due to this pre-incubation of the active ingredient in a retaining means the recovery rate of the active ingredient in the final patch formulation is significantly increased compared to the known processes. The recovery rate according to the invention is at least 20%, favourably at least 50% or 70%. Most preferably the recovery rate is at least 80% or even 100%. In this context, the term incubation or pre-incubation preferably refers to a stage in which the active ingredient is contacted with the retaining means to form a composition before this composition is applied to a pre-existing layer of the polymer matrix. The composition preferably does not contain material identical to polymer matrix to which the composition is to be applied.

From the above said it is apparent that the polymer matrix (carrier material) is not a backing layer or a release layer.

Preferably, before the composition comprising the active ingredient is applied to the polymer matrix (carrier material), the polymer matrix is dry. This preferably means that, before applying the composition, the polymer matrix is touch dry, more preferably that it contains less than 2%, 1%, 0.5%, 0.2% or 0.1% or does not contain any solvent, and even more preferably that it contains more than 98%, 99%, 99.5%, 99.8% or 99.9% or only solid components. In a preferred embodiment, the carrier material is a dried layer of polymer matrix, preferably a dried layer of adhesive polymer matrix.

The objective of the present invention can be achieved, in contrast to the standard hot melt technique and in contrast to the standard solvent/casting/drying process, by a method in which at first the active pharmaceutical ingredient is left out when forming the layer containing the polymer material and instead the active pharmaceutical ingredient is applied afterwards. In particular, the active pharmaceutical ingredient should not be present during the high-temperature drying step required for the carrier material (polymer matrix). According to the invention, in particular by applying the active pharmaceutical ingredient to a dry polymer matrix, it is possible to avoid subsequent drying at increased temperatures. Thus, in the method according to the invention preferably no drying at a temperature higher than 50° C., 45° C., 40° C. or 35° C. is performed after the composition comprising the active pharmaceutical ingredient has been applied to the carrier material. Preferably, for the active pharmaceutical ingredient either a solvent is chosen that evaporates well at room temperature or a manufacturing process is chosen that does not require drying.

The composition provided by contacting the active pharmaceutical ingredient with the retaining means either migrates into the carrier material, preferably completely, or remains on its surface. The substrate layer formed may—preferably in the case when a liquid retaining means is used—result from the mentioned composition blending with (e.g. soaking into) the carrier material so that the composition and the carrier material together form a single homogeneous or substantially homogeneous substrate layer. Alternatively, the substrate layer formed may—preferably in the case when a solid retaining means is used—result in an extra layer, which is separate or substantially separate from the carrier material.

Advantageously the transdermal patch according to the invention containing the retaining means does not require any further additive such as penetration enhancers or penetration regulators to provide a sufficient penetration of the active ingredient into the skin.

The method according to the invention is suitable for any active ingredients or mixtures thereof. However, it is preferably used to prepare transdermal patches with volatile active pharmaceutical ingredients, in particular carbamates such as rasagiline or its derivatives or analogues, preferably in the form of the free base. A volatile active pharmaceutical ingredient is any drug substance either with a melting point at room temperature of below 100° C. and/or which evaporates at a temperature of more than 30° C., more than 50° C. or more than 70° C.

The active pharmaceutical ingredient used in the present invention preferably has a chemical structure according to one of the following formulas:

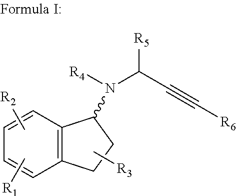

wherein
R₁ is hydrogen, halogen, alkyl, alkoxy, acyl, acyloxy, aryl, aralkyl, hydroxy, carboxy, amine, alkylamine, dialkylamine, nitro, or —OC(O)NR₁₂R₁₃, and may be substituted by one or more substituents selected from alkyl, halogen, hydroxy, carboxy, amine, alkylamine, dialkylamine;
R₂, R₃, R₄, R₅, R₆, R₁₂ and R₁₃ independent from each other are hydrogen, halogen or alkyl.

One or more, preferably one or two, of the carbon atoms in the formula including the substituents may be replaced by a heteroatom such as nitrogen, oxygen or sulfur. Preferably, R₁ is hydrogen or —OC(O)NR₁₂R₁₃, wherein R₁₂ and R₁₃ preferably are methyl and/or ethyl, more preferably R₁₂ is methyl and R₁₃ is ethyl. R₂, R₃, R₄, R₅ and R₆ each preferably are hydrogen.

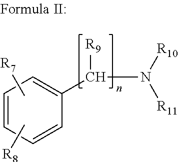

wherein
R₇ is hydrogen, halogen, alkyl, alkoxy, acyl, acyloxy, aryl, aralkyl, hydroxy, carboxy, amine, alkylamine, dialkylamine, nitro, or —OC(O)NR₁₄R₁₅, and may be substituted by one or more substituents selected from alkyl, halogen, hydroxy, carboxy, amine, alkylamine, dialkylamine;
R₈, R₉, R₁₀, R₁₄ and R₁₅ independent from each other are hydrogen, halogen or alkyl, wherein if more than one R₉ is present, these R₉ groups may also be different from each other;
R₁₁ is hydrogen, halogen or alkyl optionally substituted by halogen or hydroxy;
n is an integer from 1 to 4, preferably 1 or 2.

One or more, preferably one or two, of the carbon atoms in the formula including the substituents may be replaced by a heteroatom such as nitrogen, oxygen or sulfur. Preferably, R₇ is hydrogen or —OC(O)NR₁₄R₁₅, wherein R₁₄ and R₁₅ preferably are methyl and/or ethyl, more preferably R₁₄ is methyl and R₁₅ is ethyl. R₉ preferably is hydrogen. R₉ preferably is hydrogen or methyl. n preferably is 1 or 2. The group —[CHR₉]ₙ— preferably is —CH₂—CH(CH₃)— or —CH(CH₃)—. R₁₀ preferably is 2-propynyl (—CH₂—C≡CH) or methyl. R₁₁ preferably is methyl.

Racemic mixtures as well as isolated stereochemical forms of the above structures are encompassed by the formulas, including any isomers, stereoisomers, diastereoisomers and enantiomers.

Halogen preferably refers to fluorine, chlorine, bromine or iodine. Alkyl refers to a saturated or partially unsaturated, linear or branched carbohydrate chain preferably comprising 1 to 20 carbon atoms, more preferably 1 to 10, or 1 to 6 carbon atoms, and most preferably 1 to 4 carbon atoms. Alkyl includes unsaturated carbohydrate chains, i.e. alkenyl and alkynyl, having one or more carbon-carbon double bonds and/or one or more carbon-carbon triple bonds. Examples of alkyl are methyl, ethyl, n-propyl, isopropyl, n-butyl, tert. butyl, pentyl, hexyl, vinyl, and 2-propynyl. Alkoxy refers to the group —O-alkyl. Acyl refers to —C(O)-alkyl. Acyloxy refers to the group —OC(O)-alkyl. Aryl refers to an aromatic carbocyclic group preferably having 6 to 20 carbon atoms, more preferably 6 to 10 carbon atoms, having a single ring or multiple rings or multiple condensed rings. Examples are phenyl, naphthyl and biphenyl. Aralkyl refers to the group -alkyl-aryl.

The active pharmaceutical ingredient preferably is rasagiline or a derivative or analogue thereof. Exemplary derivatives or analogues of rasagiline are:

Ladostigil (TV3326, [(N-propargyl)-(3R)-aminoindan-5-yl]-ethyl methyl carbamate)

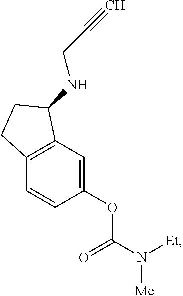

TV3279 [(N-propargyl)-(3S)-aminoindan-5-yl]-ethyl methyl carbamate

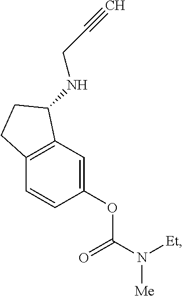

Selegiline (R)—N-methyl-N-(1-phenylpropan-2-yl)prop-2-yn-1-amine

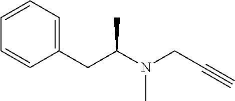

### Rivastigmine

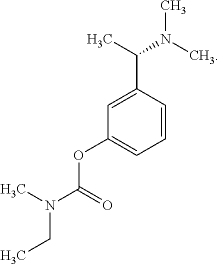

The retaining means are defined as any substance matter which either chemically or physically interacts with the active ingredient and therewith or thereby prevents its evaporation or sublimation from the polymer matrix, in particular during the manufacturing process. Preferred is a substantial interaction with the active ingredient. “Preventing evaporation or sublimation” in the context of the invention means, that the recovery rate of the active ingredient at room temperature and after the manufacture process is at least 20%, favourably at least 50% or 70%. Most preferably the recovery rate is at least 80% or even 100%.

The retaining means may be a solid or liquid substance having e.g. the active pharmaceutical ingredient attached or bound to its surface or incorporated into its body.

Suitable liquid retaining means are e.g. selected from the group consisting of natural or synthetic oils, fatty alcohols such as e.g. polidocanol, isopropyl myristate, polyalkoxylated fatty acids and polyalkoxylated fatty alcohols (in particular polyethoxylated fatty acids and fatty alcohols) with lower molecular weight, i.e. a molecular weight which renders them liquid at room temperature, and mixtures thereof. Suitable liquid retaining means have a low vapour pressure. Preferably they are compatible with the carrier material to which they are to be applied.

Suitable solid retaining means are selected e.g. from the group consisting of Eudragit polymers (such as Eudragit EPO), PVP, PEO, PVA, cellulose or derivatives, starch or derivatives, PVPVA or their blends, polyalkoxylated fatty acids and polyalkoxylated fatty alcohols (in particular polyethoxylated fatty acids and fatty alcohols) with higher molecular weight, i.e. a molecular weight which renders them solid at room temperature, and mixtures thereof. Solid and liquid retaining means can also be mixed. The physical state of the retaining means (liquid/solid) is well known to a person skilled in the art being a function of molecular weight.

When solid retaining means are used, the step of contacting an active pharmaceutical ingredient with a retaining means to provide a composition preferably involves preparing a solution or suspension comprising the active pharmaceutical ingredient and the retaining means in a highly volatile solvent (such as acetone, ethyl acetate or cyclohexane). In such a case, the method for preparing the transdermal patch preferably comprises the step of drying the formed substrate layer of the transdermal patch. The use of a highly volatile solvent allows for drying at low temperatures, preferably lower than 50° C., 45° C., 40° C. or 35° C. Preferred temperature ranges are e.g. 20 to 50° C., 20 to 45° C., 20 to 40° C., 20 to 35° C., 25 to 45° C., 25 to 40° C., 25 to 35° C., 30 to 40° C. or 30 to 35° C.

Solid retaining means allow for drying the substrate layer at room temperature, leading to a decreased loss of active pharmaceutical ingredient. Solid retaining means also allow for precise manufacturing of the transdermal patch. It is possible to manufacture the respective solid layers separately and to join the finished layers afterwards, e.g. by laminating. This results in a lower variation of the desired layer thickness as compared to the case when multiple layers are manufactured directly on top of each other.

The retaining means may be covered or coated with the active pharmaceutical ingredient in any manner known in the art, for example by applying the active pharmaceutical ingredient in a molten state and solidifying it, by applying a solution of the active pharmaceutical ingredient and drying it, or by any other method. The incorporation of the active pharmaceutical ingredient into the retaining means may be achieved by melting both materials, mixing them and cooling the mixture, or by solving both materials in the same solution and drying (e.g. spray-drying) the solution. The resulting composition can then be applied to the polymer matrix.

In a preferred embodiment the retaining means is a liquid with a low volatility (“low volatile solvent”). This is defined as any substance that is liquid from room temperature up to 70° C.

As a carrier material (“polymer matrix”) any common or suitable material may be used. The mixture including the active ingredient and the retaining means are added to the polymer matrix to form the “substrate layer” of the final transdermal patch.

Preferably, the carrier material comprises at least one type of the following polymer materials: polyacrylates and derivatives thereof, silicone polymers and derivatives thereof, polyisobutylene (PIB) and derivatives thereof, ethylene-vinyl acetate copolymers and derivatives thereof, Styrene-block-co-polymers and derivatives thereof, polyacrylic acids and derivatives thereof, polyoxazolines (POX) and derivatives thereof, poylurethanes and derivatives thereof, polyolefines and derivatives thereof, polyesters and derivatives thereof.

The carrier material may be adhesive, thus constituting the support layer for the active ingredient as well as the adhesive layer for the adherence of the patch to the skin. It can also be non-adhesive. Then a separate adhesive layer has to be incorporated into the transdermal patch.

In one embodiment of the invention, the retaining means may be a liquid. In this case, the active pharmaceutical ingredient can be solved or dispersed first in the retaining means, forming a solution or suspension. Then the carrier matrix is soaked with the solution or suspension.

To aid the soaking process, well known soaking additives such as Plastoid B, Eudragit polymers, SiO₂, PEO, PVP, PVA, cellulose or derivatives, starch and derivatives, cyclodextrins and the like, either alone or in combination may be added to the carrier material or to the solution/suspension comprising the active pharmaceutical ingredient (drug). A soaking additive added to the carrier may help in preventing softening of the carrier. Softening on the one hand increases an adhesive force, but also results in undesired properties such as cold flow or stringing effects during removal of the release liner. A soaking additive added to the solution or suspension may help in increasing the viscosity, however the solution or suspension should not be too viscous in order to ensure efficient soaking. Furthermore, the solution or suspension may be made viscous by adding an additive such as a non-adhesive carrier material and may then be applied to the surface of the carrier matrix. It is advantageous if the solution/suspension is viscous enough to allow knife-coating. This technique is widely used for the preparation of transdermal patches, so that standard equipment may be used for such solutions/suspensions, which allows for a more economical manufacturing process. Additionally, it is more precise than spraying, which furthermore may be hazardous due to created aerosols.

When a liquid retaining means is used, it is preferred to choose an adhesive carrier material compatible to the solution/suspension to be applied, so that no phase separation occurs.

In preferred embodiments, the solid composition is ground. The ground material may then be dispersed in a melt or solution of the carrier material, followed by the cooling or drying of the mixture to form the substrate layer of the transdermal patch. Alternatively, the solid composition comprising the retaining means and the active pharmaceutical ingredient may be applied to the solid carrier material being adhesive. Here, the composition may either be applied as granulate material or powder (e.g. after grinding) or as bulk material (e.g. as fleece material).

The transdermal patch prepared by the methods according to the invention may further comprise a backing layer, preferably an inert backing layer, i.e. a backing layer which does not interfere with the activity or bioavailability of the active pharmaceutical ingredient, a protective layer, i.e., e.g., a layer protecting the bottom of the transdermal patch during storage which has to be peeled off prior to use, and/or a release controlling layer which regulates the rate of release of the active ingredient. The backing layer (overlay) can e.g. be a foil or a film of polyethylene or polypropylene or a non-woven fabric. The protective layer (antistick layer, release liner) can also be a foil, or a complex film formed by materials such as polyethylene or polypropylene or polycarbonate, etc, or a thick slick paper pretreated with paraffin oil or siliconized or fluoro coated. The release controlling layer may be a membrane having a defined pore size and may be used for slowing the release and/or achieving a release that has an extended linear phase.

The transdermal patch preferably comprises an adhesive layer for contacting the skin, which is adjacent to the substrate layer or adjacent to an optionally present release controlling layer. This layer is preferably added a certain time (e.g. 10 to 60 min, preferably 20 to 40 min) after the composition comprising the active pharmaceutical ingredient has been applied to the carrier material, during which resting step the assembly is preferably kept at room temperature. In the case of using a liquid retaining means, the resting step is advantageously performed so that the composition is allowed to migrate into the carrier material. Adding the adhesive layer is preferably done by laminating. Such an adhesive layer adjacent to the substrate layer may be particularly useful when using a certain liquid retaining means such as an oily liquid retaining means, because oil may be present on the surface of the soaked layer, which might result in an impaired adhesiveness. An adhesive layer may also be particularly useful when the substrate layer (or the optionally present release controlling layer) is not sufficiently adhesive.

In one embodiment of the invention, the retaining means is a low volatile liquid plasticizer and the carrier material is a mixture of an adhesive polymer and a non-adhesive polymer. The carrier material preferably is chosen such that it exhibits a rather low adhesiveness. Upon soaking of the carrier material with the retaining means comprising the active pharmaceutical ingredient, the carrier material is softened by the plasticizer and its adhesiveness is increased to a suitable level.

In preferred embodiments, no penetration enhancer in addition to the retaining means—which itself may be a penetration enhancer—is added to the transdermal patch. More preferably no penetration enhancer at all is added to the transdermal patch. At times it is advantageous if the transdermal patch does not contain further additives such as metal salts.

In the following, particular embodiments of the method according to the invention are described:

One embodiment comprises the steps of
a) adsorbing the active pharmaceutical ingredient onto the solid retaining means to form a composition, preferably by melting the active pharmaceutical ingredient in presence of the retaining means and then solidifying;
b) grinding the composition;
c) mixing the ground composition with molten carrier material or with a solution comprising the carrier material;
d) applying the dispersion or solution onto a backing layer to form a film; and
e) cooling or drying the film to form a substrate layer.

In a further embodiment the method comprises the steps of:
a) forming an adhesive matrix layer comprising the carrier material, e.g. by coating or laminating;
b) mixing the active pharmaceutical ingredient with the liquid retaining means to form a solution or suspension;
c) applying the solution or suspension to the matrix layer to form a substrate layer, preferably by soaking, printing or casting.

In yet another embodiment the method comprises the steps of:
a) forming an adhesive matrix layer comprising the carrier material, e.g. by coating or laminating;
b) mixing the active pharmaceutical ingredient with the liquid retaining means and a non-adhesive carrier material to form a viscous solution or suspension;
c) applying the solution or suspension to the matrix layer to form a substrate layer, preferably by spreading; and
d) optionally applying another adhesive layer.

In a further embodiment the method comprises the steps of:
a) forming an adhesive matrix layer comprising the carrier material, e.g. by coating or laminating;
b) attaching the solid retaining means, preferably a fleece material, to the adhesive matrix layer;
c) applying the active pharmaceutical ingredient to the retaining means, for example by impregnating, either before or after the retaining means is attached to the adhesive matrix layer; and
d) applying another adhesive layer to the other site of the retaining means.

In another embodiment the method comprises the steps of:
a) forming an adhesive matrix layer comprising the carrier material, e.g. by coating or laminating;
b) adsorbing the active pharmaceutical ingredient onto the solid retaining means to form a composition, preferably by melting the active pharmaceutical ingredient in presence of the retaining means and then solidifying;
c) grinding the composition;
d) applying the ground composition to the adhesive matrix layer to form a substrate layer; and
e) optionally applying another adhesive layer.

In yet another embodiment the method comprises the steps of:
a) forming an adhesive matrix layer comprising the carrier material, e.g. by coating or laminating;
b) applying the active pharmaceutical ingredient to the solid retaining means to form a composition by mixing the active pharmaceutical ingredient and the retaining means in the presence of a highly volatile solvent;
c) applying the composition to the adhesive matrix layer to form a substrate layer;
d) drying the substrate layer; and
e) optionally applying another adhesive layer.

Furthermore, the present invention relates to a method for preparing a transdermal patch comprising a substrate layer, the method comprising the steps of:
a) melting a carrier material;
b) quickly dispersing an active pharmaceutical ingredient into the molten carrier material;
c) forming a substrate layer with the mixture obtained in step (a) or (b); and
d) quickly cooling the substrate layer to solidify it.

In this method, the carrier material preferably is an adhesive, the substrate layer preferably is formed on a backing layer and/or the substrate layer preferably is cooled by forming it on a cooled surface. Furthermore, each of the specific embodiments described herein with respect to the methods for preparing a transdermal patch also apply to this method, if appropriate.

In another aspect, the present invention provides a transdermal patch comprising a backing layer and a substrate layer comprising a volatile active pharmaceutical ingredient, a carrier material and a retaining agent. Preferably, the transdermal patch is obtainable or prepared by any one of the methods according to the invention. Furthermore, the embodiments described herein with respect to the preparation methods also apply accordingly to the transdermal patch according to the invention.

In preferred embodiments, the transdermal patch does not contain a penetration enhancer in addition to the retaining means which itself may be a penetration enhancer. More preferably, the transdermal patch does not contain any penetration enhancer at all.

The transdermal patch preferably contains at least 1% (w/w) of the retaining means. Furthermore, it preferably contains at least 0.1% (w/w) of the active pharmaceutical ingredient.

Preferred prototypes are manufactured as follows:

Prototype 1 (see FIG. 9):

An acrylate adhesive layer (without active pharmaceutical ingredient) is coated or laminated to a backing layer. Rasagilin base is dissolved in Plastoid B and neutral oil and directly coated to the dry acrylate adhesive layer. The assembly is allowed to rest at 30° C. or below, e.g. at room temperature so that the rasagilin base/Plastoid B/neutral oil mass may diffuse into the acrylate layer. After this, a second, dry acrylate layer is added by laminating. The final laminate is a sandwich.

Prototype 2 (see FIG. 10 A):

An acrylate adhesive layer (without active pharmaceutical ingredient) is coated or laminated to a backing layer. Rasagilin base is dissolved in Eudragit EPO and acetone and directly coated to the dry acrylate adhesive layer. A preferred alternative is coating the rasagilin base/Eudragit EPO/acetone mass onto a foil. After coating with the rasagilin base/Eudragit EPO/acetone mass, drying is done at 30° C. or below, e.g. at room temperature. This is possible due to the high vapour pressure of acetone. The result is a solid, non-adhesive rasagilin base/Eudragit EPO layer. A second, dry acrylate layer is added by laminating. The final laminate is a sandwich.

Prototype 3 (see FIG. 10 B)

Prototype 3 is manufactured like prototype 2, however using a membrane. Thus a further acrylate adhesive layer is necessary.

In one embodiment, the transdermal patch according to the invention is for use in medicine. In particular, it contains rasagiline or a derivative thereof as the active pharmaceutical ingredient and is for treatment or prophylaxis of a nervous system disease, preferably a nervous system disease selected from the group consisting of Parkinson's disease, Alzheimer's disease, depression, hyperactive child syndrome, restless leg syndrome, multiple sclerosis and abstinence syndrome.

Thus, in one specific embodiment the invention provides a rasagiline transdermal patch for treatment or prophylaxis of nervous system diseases, wherein the patch comprises an inert backing layer chemically inert to substrate ingredients, a substrate layer comprising the free base of rasagiline and a protective layer to be peeled off before use, wherein the substrate layer is a drug-carrying reservoir comprising an organic polymer that is soaked with pharmaceutically acceptable low volatile solvent containing rasagiline or that is dispersed with a ground absorber material comprising rasagiline.

The inventors have found that that a transdermal patch (TDS) comprising an organic matrix which is soaked with a low volatile solvent solubilising the rasagiline permits a highly efficient skin penetration. Furthermore, rasagiline in TDS formulations according to this invention exhibits the stability that is required for a pharmaceutical formulation.

This skin penetration was tested in an in vitro method using excised human skin, representing a model that is highly relevant for the human clinical situation.

Due to the use of a low volatile solvent a subsequent drying step is not necessary. This results in a more efficient production process and prevents the rasagiline and the patch material from drying-associated damage especially as result of the high drying temperature.

The low volatile solvent can also act as an enhancer for the transdermal absorption of rasagiline. If appropriate, the transdermal patch may further comprise a controlled release substrate layer, a release controlling layer, such as a membrane having a defined pore size and/or an adhesive layer. If the drug reservoir substrate layer or the controlled release substrate layer has an appropriate adhesiveness, the adhesive layer as a separate layer may be unnecessary.

Furthermore, a rasagiline transdermal patch is provided wherein the substrate layer comprises a ground layer containing an organic polymer coated with a high viscosity layer comprising a pharmaceutically acceptable low volatile solvent containing rasagiline.

In the above rasagiline transdermal patch, rasagiline has an effective amount of 0.01 mg/cm² to 50 mg/cm² in the TDS,

The rasagiline transdermal patch of the present invention has an administration area of about 1 cm² to 50 cm² during a transdermal therapy.

Throughout this application certain trademarks for chemicals are mentioned. This is in each case meant to be a reference to the following generic groups of chemicals, respectively, as long as they fulfil the respective function to a sufficient degree:

| Trademark | Generic group of chemicals |
|---|---|
| Plastoid B | Poly(butylmethacrylat-co-methylmethacrylate) |
| Eudragit EPO and | Aminoalkyl methacrylate copolymer |
| Eudragit | |
| Cremophor EL | Polyoxyethylenglyceroltriricinoleate 35 |
| Brij S20 | Macrogol Stearyl Ether |
| Brij CS20 | PEG-20 Cetostearyl ether |
| Crodet S40 | PEG-40 Stearate |
| Cithrol 10 MS | PEG-20 Stearate |
| Miglyol 812 | Caprylic/Capric Triglycerides |
| Hostaphan RN15 | PET foil (15 μm) |
| FL2000 | Siliconized PET foil (100 μm) |
| DuroTak 87-4287 | Polyacrylate adhesive |
| DuroTak 87-611 | Styrenic rubber adhesive |
| DuroTak 87-618A | Polyisobutylene adhesive |
| BioPSA | Silicone adhesive |

However, in these cases the specific chemicals covered by the trademark at the filing date of this application or closely related chemicals are preferred.

The following figures and examples serve the purpose to illustrate the present invention without in any way limiting this scope thereof. However, they relate to preferred embodiments of the present invention.

### BRIEF DESCRIPTION OF THE DRAWINGS

FIG. 1 shows the time-cumulative penetration of a saturated solution of the free base of rasagiline across excised human skin (EHS). A saturated solution of rasagiline base in phosphate buffer pH 6.5 (No 4010800, Ph. Eur. 6.0) was prepared and ist permeation across excised human skin was tested

FIG. 2 shows the loss of rasagiline substance during state of the art patch manufacturing methods as revealed by low skin permeation rates (EHS).

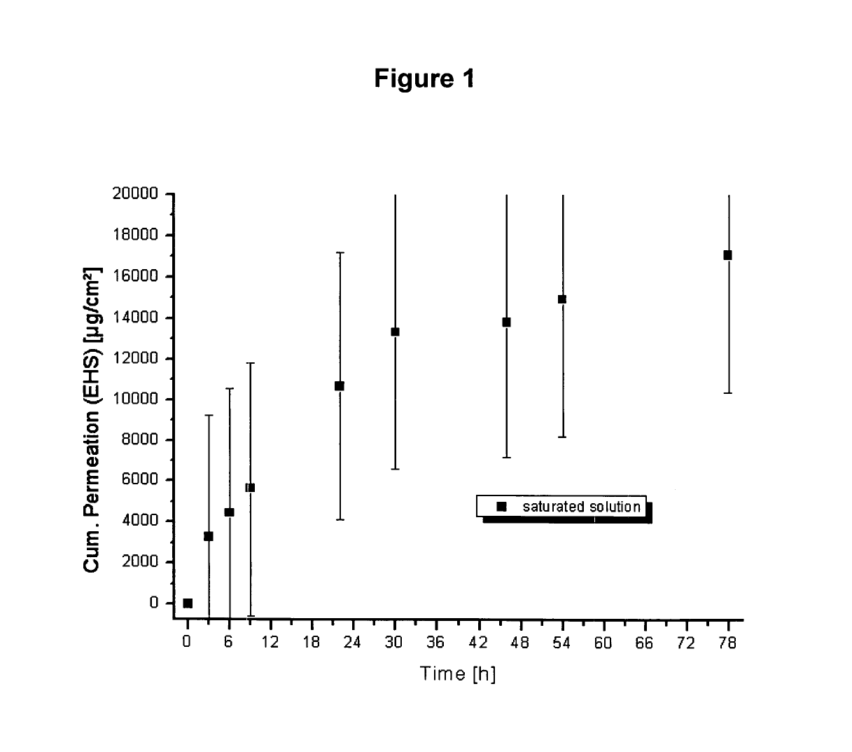

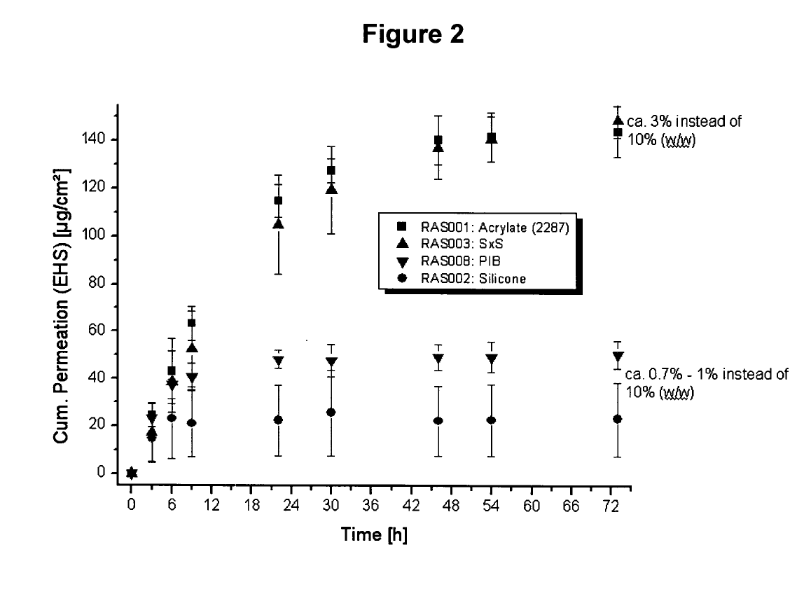

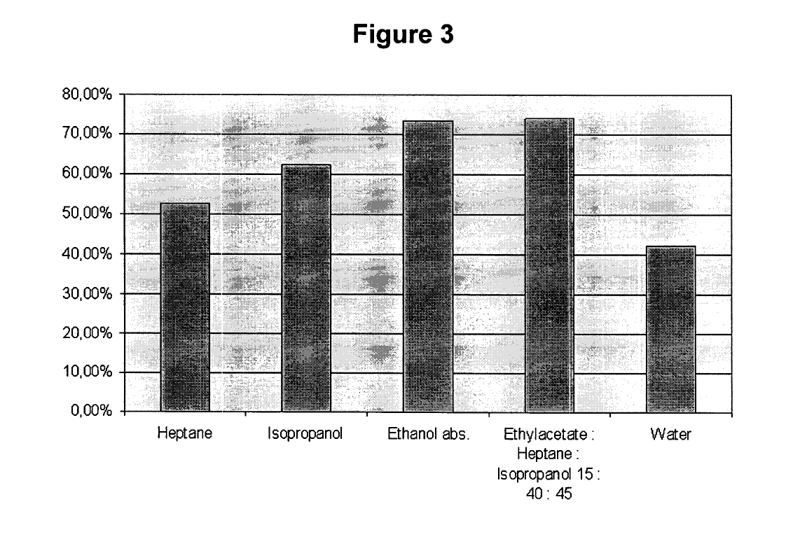

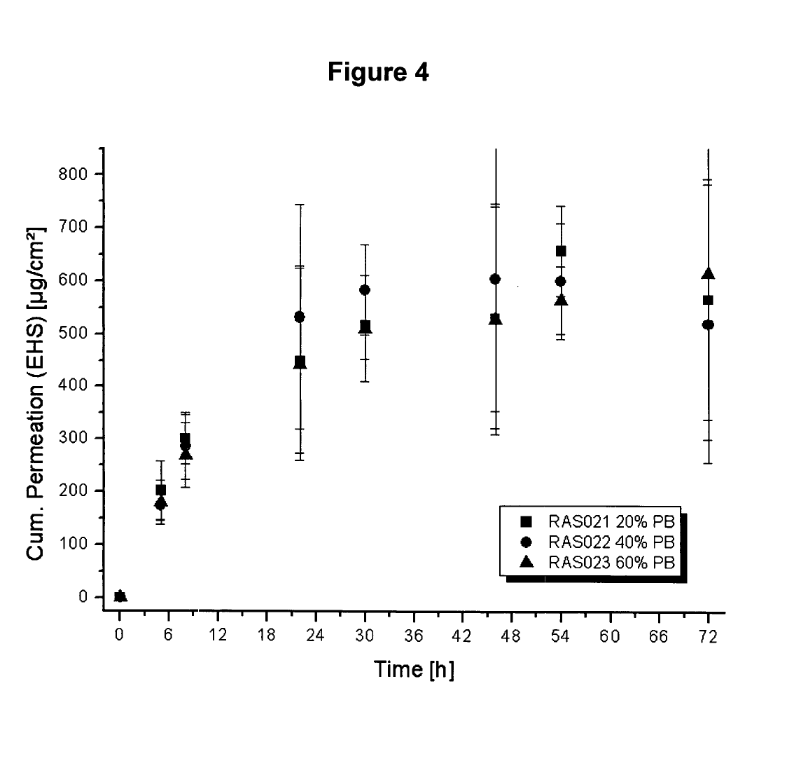

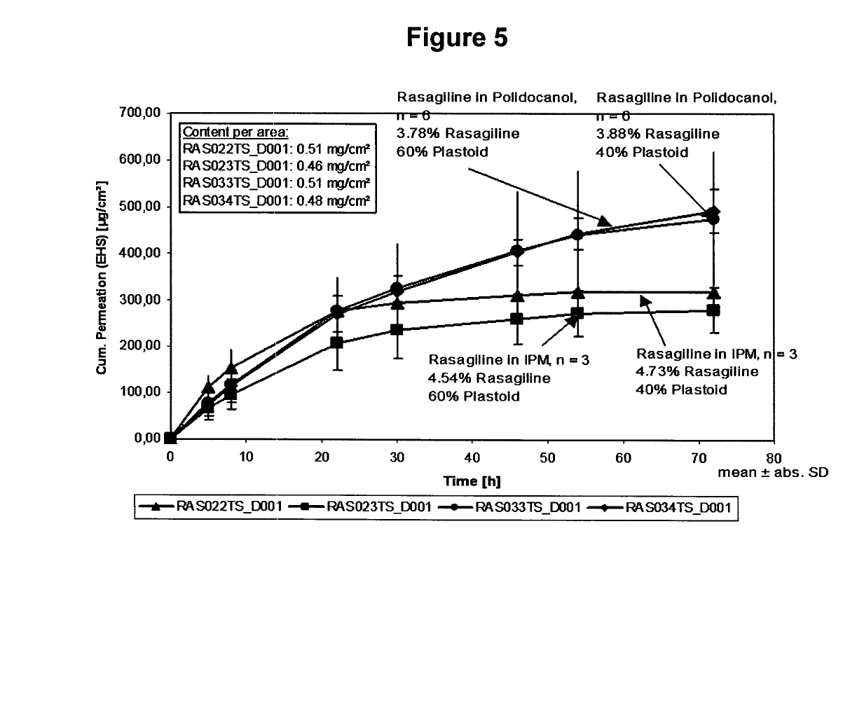

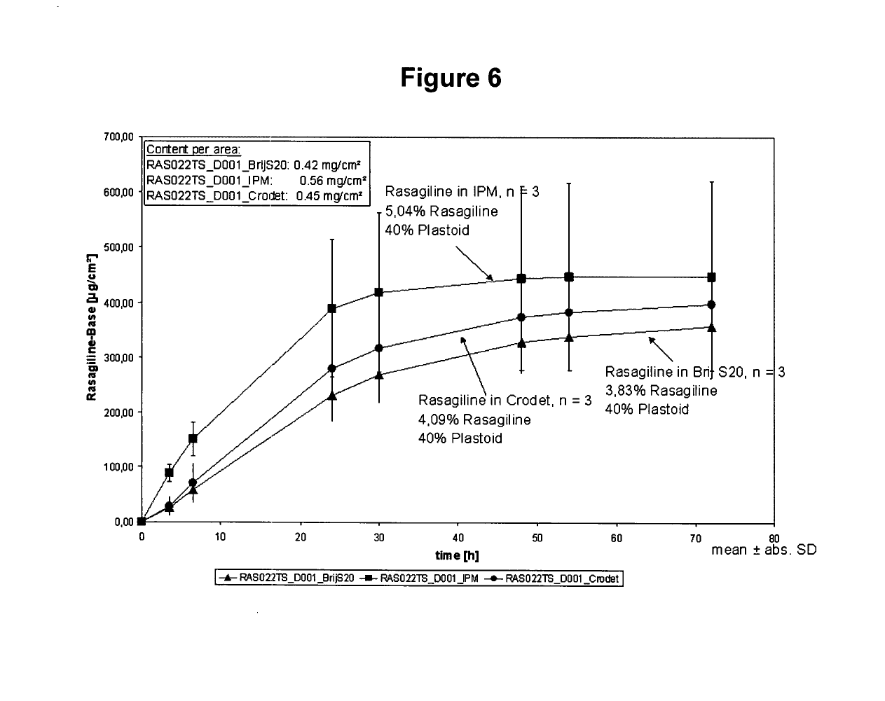

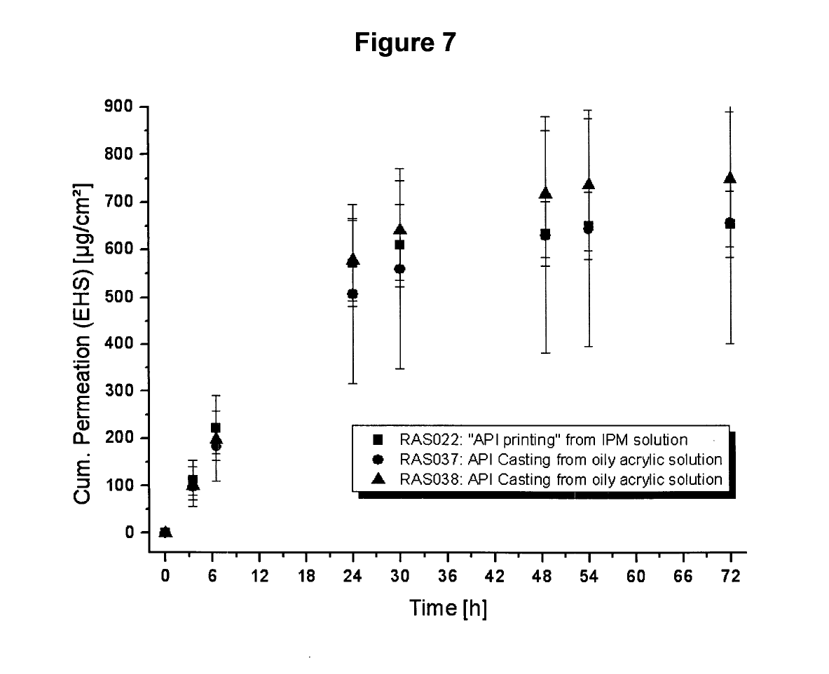

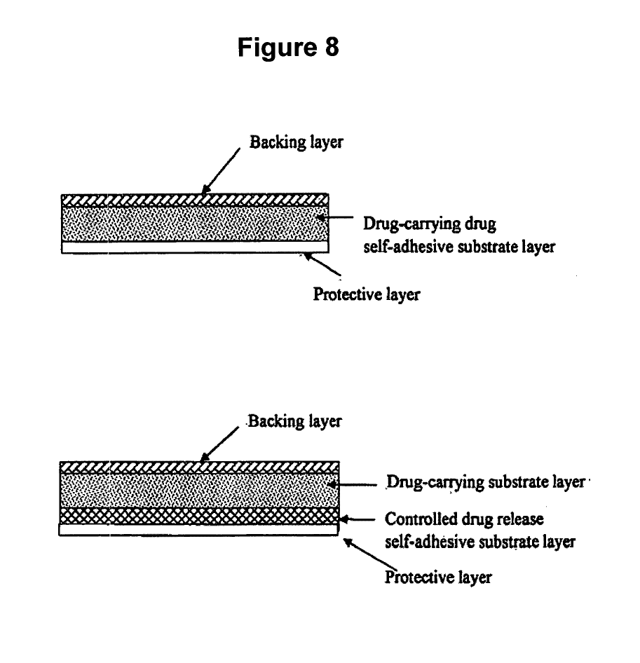

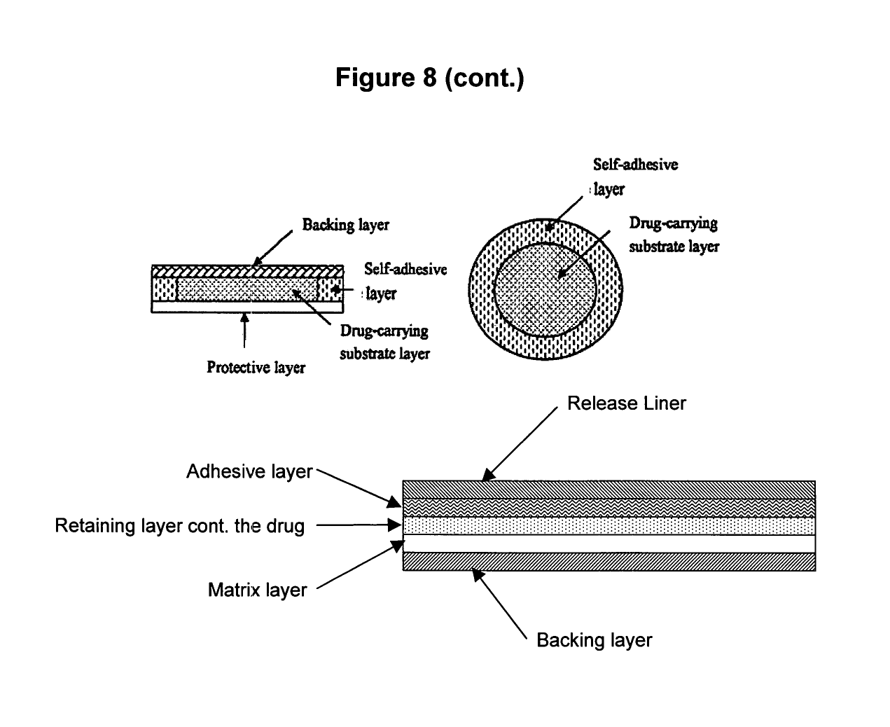

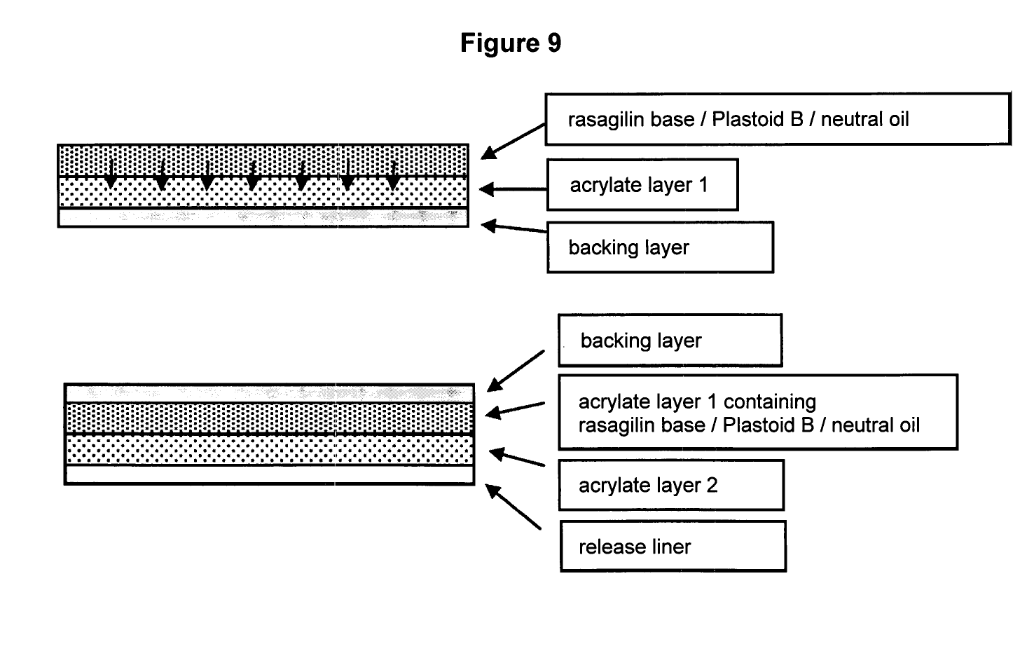

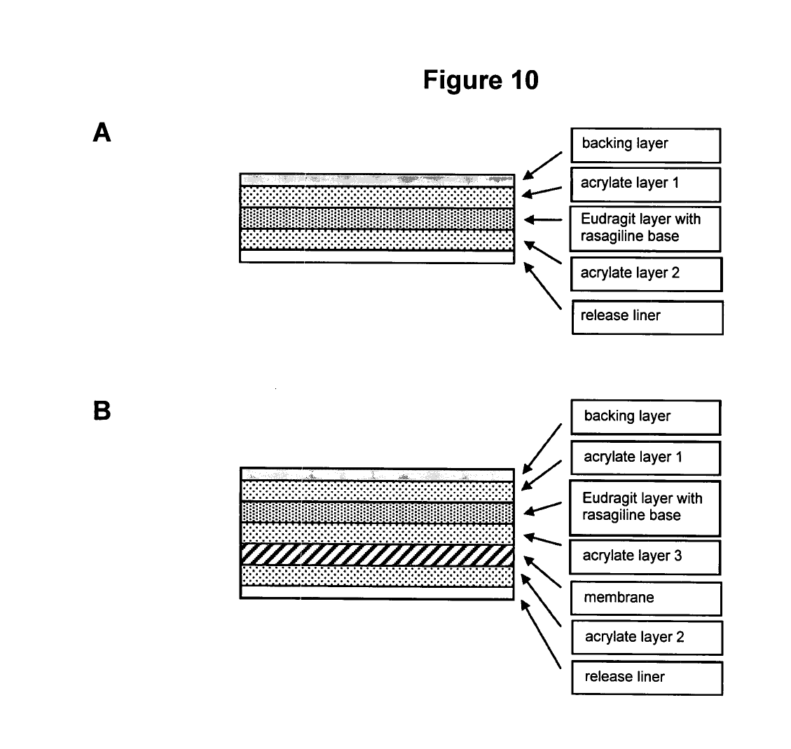

Acrylate: Rasagiline base was dissolved in ethyl acetate and added to a solution of durotak 2287 (acrylic adhesive), the mixture was cast to a liner, dried and laminated with a protective foil to form a standard solvent casting acrylate TDS

Silicone: Two Silicone adhesive solutions (BioPSA 7-4301/7-4201) were mixed. An ethanolic PVP solution was mixed with rasagiline base. This mixture was added to the silicone adhesived solution under stirring, cast onto a liner, dried, covered with a protective liner to form a standard solvent casting silicone TDS

SxS (heptane): Rasagiline base was dissolved in heptane and added to a solution of DuroTak 611a under stirring, cast onto a liner, dried, covered with a protective liner to form a standard solvent casting SxS TDS.

PIB: Rasagiline base was dissolved in ethanolic PVP solution and added to a mixture of high, medium and low MW PIB in hexan under stirring, cast onto a liner, dried, covered with a protective liner to form a standard solvent casting PIB TDS

FIG. 3 shows the loss of rasagiline substance in % of starting material after removal of different solvents by drying. Rasagiline base (50 mg) was dissolved in the presented solvents (0.14 g) and the placed into an over at 70° C. to monitor its loss. Surprisingly, even pure water, which is not a preferred solvent of a lipophilic base cause significant sublimation (=loss) of rasagiline.

FIG. 4 shows the time-cumulative EHS penetration of a transdermal patch with an acrylate matrix containing 20% (▪), 40%, () or 60% (▴) of Plastoid B and rasagiline solubilised in the low volatile solvent isopropyl myristate. An acrylic adhesive matrix comprising Durotak 2287 further containing either 20, 40 or 60% (w/w) of Plastoid B as absorber were prepared by solvent casting. Rasagiline base solutions were prepared and applied to the above described matrix, after complete soaking of this solution into the matrix, a protective liner was added. A solution of Rasagiline was prepared by dissolving Rasagiline base at room temperature with IPM as solvent. Samples were then tested for rasagiline base permeation from these patches across excised human skin.

FIG. 5 shows the time-cumulative EHS penetration of a transdermal patch containing:
(i) 3.78% rasagiline in polidocanol and 60% of Plastoid B (♦);
(ii) 3.88% rasagiline in polidocanol and 40% of Plastoid B ();
(iii) 4.54% rasagiline in isopropyl myristate and 60% of Plastoid B (▴);
(iv) 4.73% rasagiline in isopropyl myristate and 40% of Plastoid B (▪).

An acrylic adhesive matrix comprising Durotak 2287 further containing either 40 or 60% (w/w) of Plastoid B as absorber were prepared by solvent casting. Rasagiline base solutions were prepared and applied to the above described matrix, after complete soaking of this solution into the matrix, a protective liner was added. A solution of Rasagiline was prepared by dissolving Rasagiline base at room temperature with polidocanol as solvent versus IPM as solvent. Samples were then tested for rasagiline base permeation from these patches across excised human skin.

FIG. 6 shows the time-cumulative EHS penetration of a transdermal patch produced by a “printing process” and containing:
(i) 5.04% rasagiline in isopropyl myristate and 40% of Plastoid B (▪);
(ii) 4.09% rasagiline in Crodet and 40% of Plastoid B ()
(iii) 3.83% rasagiline in Brij S20 and 40% of Plastoid B (▴)

An acrylic adhesive matrix comprising Durotak 2287 further containing either 40% (w/w) of Plastoid B as absorber were prepared by solvent casting. Rasagiline base solutions were prepared and applied to the above described matrix, after complete soaking of this solution into the matrix, a protective liner was added. A solution of Rasagiline was prepared by dissolving Rasagiline base at room temperature with IPM as solvent versus Brij or Crodet at slightly elevated temperature as solvent. Samples were then tested for rasagiline base permeation from these patches across excised human skin.

FIG. 7 shows the time-cumulative EHS penetration of a transdermal patch produced by a printing process using an isopropyl myristate solution (▪) or by a casting process using an oily acrylic solution (; ▴).

FIG. 8 shows exemplary cross section views of the patches of Examples 1-6.

FIG. 9 shows cross section views of the patch of Prototype 1 as described above in two different manufacturing stages.

The top view shows the stage in which the applied rasagilin base/Plastoid B/neutral oil mass diffuses into the acrylate layer. The bottom view shows the final laminate.

FIG. 10 shows cross section views of the final laminates of the patch of Prototype 2 (FIG. 10 A) and of Prototype 3 (FIG. 10 B).

### EXAMPLES

### Example 1

### Pilot Test Regarding the Solubility of Rasagiline Base in Non-Volatile Solvents for Use in a TDS

Methods and Results

1.1 Solubility of Rasagiline Base

| | 0.5 g or mL, respectively | 1.0 g or mL, respectively |
|---|---|---|
| Weighed sample ca. | Equates to a ca. 1:20 | Equates to a ca. 1:40 |
| 20-25 mg | mixture | mixture |
| Isopropyl myristate | Solubilised | Solubilised |
| (IPM) | | |
| Cremophor EL | Nearly completely | Solubilised |
| | solubilised | |
| Vitamin-E-acetate | Nearly completely | Solubilised |
| | solubilised | |
| Propylenglycol | Solubilised | Solubilised |
| Silicone oil 1000 | Not Solubilised | Solubilised |
| PEG 400 | Solubilised | Solubilised |
| Glycerol anhydrous | Not Solubilised | Not completely |
| | | solubilised, |
| | | residual crystals |
| | | observable |
| paraffine, viscous | Not Solubilised | Solubilised |
| Polysorbat 80 | Solubilised | Solubilised |

1.2 Use of Isopropyl Myristate (IPM) and Different Adhesives

Goal: Compatibility of liquid retaining means and carrier materials
The adhesive is streaked and dried, a TDS 4.5 cm² is punched out
ca. 200 μl of a IPM solution containing rasagiline (Eppendorf pipet) are dropped onto it
Target concentration c(rasagiline)=5 mg/10 cm² or 2.25 mg/4.5 cm², respectively
Actual concentration c(rasagiline)=ca. 4.3 mg/4.5 cm²

| | |
|---|---|
| Durotak 87-611A | A liquid paddle remains on the patch |
| (Styrene) | |
| Durotak 87-4287 | The solution permeates into the adhesive matrix, |
| (Acrylate) | resulting in a matrix with a very soft consistency, |
| | The addition of Eudragit E hardens the matrix |
| Durotak 87-618A | A liquid puddle remains on the patch |
| (PIB) | |
| BioPSA 7-4301 | A liquid puddle remains on the patch |

1.3 Use of IPM in Combination with Acrylate/Eudragit E

Goal: Compatibility of liquid retaining means and carrier materials
Preparation of a TDS of 4.5 cm² using 20%, 40% and 60% Eudragit E in Durotak 87-4287 (RAS017, RAS018, RAS019)
ca. 50 mg of a rasagiline solution from experiment 1.1 are evenly dropped onto the TDS (with Pasteur pipet)
concentration c(rasagiline)=5 mg/10 cm² or 2.25 mg/4.5 cm², respectively
Actual concentration c(rasagiline)=ca. 1 mg/4.5 cm²

| | RAS017 | RAS018 | RAS019 |
|---|---|---|---|
| Solvent | Eudragit 20% | Eudragit 40% | Eudragit 60% |
| Isopropyl | Solution permeates | Solution | Solution |
| myristate (IPM) | into the matrix, very | permeates into | permeates |
| | soft matrix, some | the matrix, | into the |
| | residual liquid | very soft | matrix, very |
| | remains | matrix | soft matrix |

| | |
|---|---|
| Cremophor EL | Solution does not permeate but contracts |
| Vitamin-E-acetat | Solution does not permeate but contracts |
| Propylene glycol | Solution permeates, probably due to the evaporation of |
| | the propylene glycol |
| PEG 400 | Solution does not permeate but contracts |
| Glycerol, | Solution does not permeate but contracts |
| anhydrous | |
| paraffine, viscous | Solution does not permeate but contracts |
| Polysorbat 80 | Solution does not permeate but contracts |

1.4 Use of Silicone Oil in Combination with BioPSA 7-4301

Goal: Compatibility of liquid retaining means and carrier materials
BioPSA 7-4301: A TDS of 4.5 cm² is punched out
ca. 50 mg of a rasagiline solution from experiment 1.1 are evenly dropped onto the TDS (with Pasteur pipet)
Target concentration c(rasagiline)=5 mg/10 cm² or 2.25 mg/4.5 cm², respectively
Actual concentration c(rasagiline)=ca. 1 mg/4.5 cm²
Result: Solution permeates into the matrix, resulting in a very soft matrix

1.5 Further Solubility Experiments Using IPM and “Silicone Oil 1000”

Goal: Compatibility of liquid retaining means and carrier materials
rasagiline base solubilised in IPM: Solubility >200 mg rasagiline/g IPM Used concentration: 51.1 mg rasagiline in 258.4 mg IPM (higher concentration compared to Example 1.3)
Rasagiline IPM solution ca. 7 mg applied on acrylate/EPO-TDS
Target concentration c(rasagiline)=5 mg/10 cm² or 2.25 mg/4.5 cm², respectively
Actual concentration c(rasagiline)=ca. 1 mg/4.5 cm²

| | | |
|---|---|---|
| Eudragit 20% | Eudragit 40% | Eudragit 60% |

| | | |
|---|---|---|
| Isopropyl | Good permeation of the solution, | Good permeation of the |
| myristate | soft/sticky matrix | solution, firm and non- |
| (IPM) | | adhesive matrix |

rasagiline base solubilised in silicone oil: solubility ca. 25-30 mg/g silicone oil 1000
Rasagiline silicone oil solution 5 mg/10 mg/20 mg/40 mg applied onto a 4.5 cm² TDS patch (BioPSA 7-4301)
Target concentration c(rasagiline)=5 mg/10 cm² or 2.25 mg/4.5 cm², respectively
Actual concentration c(rasagiline)=ca. 0.125-1 mg/4.5 cm² (Obviously to low, ca. 90 mg are expected)

| Silicone oil 1000 | BioPSA 7-4301 |
|---|---|
| 5 mg | Sticky, not too soft |
| (0.125 mg rasagiline) | |
| 10 mg | Sticky, not too soft |
| (0.25 mg rasagiline) | |
| 20 mg | Sticky, not too soft |
| (0.5 mg rasagiline) | |
| 40 mg | Very soft matrix |
| (1 mg rasagiline) | |

1.6 Thickening of the Rasagiline IPM Solution Using Eudragit E
After addition of Eudragit E to the rasagiline solution (ca. 1:1 and 2:3) the mixture thickens, and becomes pasty, can not be streaked
Comment:
   the thickened solution should applied/laminated on the dried adhesive matrix
   for a solid matrix a cross-linked adhesive should be used (e.g. Durotak 2516)

1.7 Concentrating of Rasagiline in IPM
rasagiline base solubilised in IPM: solubility ca. 1 g/g IPM actual concentration: 50.2 mg rasagiline in 53.2 mg IPM
rasagiline is solubilised, yielding a yellow solution
Allow to stand over night: rasagiline is still solubilised, solution has a more intense yellow colour
Stirring shortly, rasagiline crystallizes from the solution
Crystals dissolve due to short heating

1.8 Application of Rasagiline IPM Solution onto Acrylate-Eudragit E-TDS

Pilot Experiment:

IPM is applied on a 10 cm² TDS, can be spread over the complete TDS

Experiment 1.8a:
1 part rasagiline is solubilised in 3 parts of IPM resulting in a concentration of 5 mg/10 cm²
The solution is stirred for 15 min
20 mg of the solution are applied onto a TDS of 10 cm², dispersed with a small spatula and the exact applied quantity is tared
The patch is allowed to stand for 30 min, so that the solution can permeate; afterwards the patch is laminated with FL 2000
RAS 017 TS_D001 with 5 mg rasagiline, 15 mg IPM n=3, the actual content can be found in the manufacturing protocol “RAS 020 TS_D001”
RAS 018 TS_D001 with 5 mg rasagiline, 15 mg IPM n=3
RAS 019 TS_D001 with 5 mg rasagiline, 15 mg IPM n=2
The patches are analysed 3 days after preparation for crystals.

Result: Only sporadic crystals were observed, macroscopically the patches exhibit a wave-like structure

Experiment 1.8b:
1 part rasagiline is solubilised in 9 parts of IPM resulting in a concentration of 5 mg/10 cm²
The solution is stirred for 15 min
20 mg of the solution are applied onto a TDS of 10 cm², dispersed with a small spatula and the exact applied quantity is tared
The patch is allowed to stand for 60 min, so that the solution can permeate; afterwards the patch is laminated with FL 2000
RAS 017 TS_D001 with 5 mg rasagiline, 45 mg IPM n=1
RAS 018 TS_D001 with 5 mg rasagiline, 45 mg IPM n=1
RAS 019 TS_D001 with 5 mg rasagiline, 45 mg IPM n=1
The patches are analysed 3 days after preparation for crystals.

Result: Only sporadic crystals were observed, macroscopically the patches exhibit a prominent wave-like structure

1.9 Application of a Rasagiline IPM Solution onto Acrylate-Plastoid B-TDS

Preparation of a 10 cm² TDS with 20%, 40% and 60% Plastoid B in Durotak 87-4287 (RAS021, RAS022, RAS023)
1 part rasagiline is solubilised in 3 parts of IPM resulting in a concentration of 5 mg/10 cm²
The solution is stirred for 15 min
20 mg of the solution are applied onto a TDS of 10 cm², dispersed with a small spatula and the exact applied quantity is tared
The patch is allowed to stand for 60 min, so that the solution can permeate; afterwards the patch is laminated with FL 2000
RAS 021 TS_D001 with 5 mg rasagiline, 15 mg IPM n=3
Result: The matrix is solid and adhesive
RAS 022 TS_D001 with 5 mg rasagiline, 15 mg IPM n=3
Result: The matrix is solid but less adhesive than RAS 021
RAS 023 TS_D001 with 5 mg rasagiline, 15 mg IPM n=3
Result: The matrix is solid but less adhesive than RAS 021
The patches are analysed 4 days after preparation for crystals.
Result: Only sporadic crystals were observed, macroscopically the patches exhibit a slight wave-like structure
From every batch 6 TDS patches with an area of 1.5 cm² are punched out and analysed for in vitro skin permeation
Result: Patch from exp. 1.9 shows superior skin permeation compared to patch from exp. 1.8. Analysis of the patches RAS021-023 in skin permeability revealed a high cumulative permeation rate for all three patches (see FIG. 4).

1.10 Sublimation of Rasagiline Base at 60-70° C.

Rasagiline is melted in the beaker glass at 60-70° C. and covered with an ice-cold watch glass

Result: The rasagiline re-sublimates on the cooled watch glass.

Conclusion:

A processing of the rasagiline in volatile/low boiling solvents is not possible. Even a slight increase in temperature results in a sublimation/evaporation of the substance.

A particularly promising strategy includes the preparation of a placebo patch with subsequent application of a rasagiline solution.

In order to realize this strategy, an additional absorbent polymer or layer has to be added to the PSA (pressure sensitive adhesive). For this purpose Plastoid B/Eudragit polymers and the like are preferably suited. Preferably, the additional polymer or layer diffuses into the placebo patch so that it does not form an additional layer in the final patch.

Particularly suitable solvents are IPM and silicone oil. However, a relatively high concentration of rasagiline in the solvent is required, preferably a conc. ≧50%, in order to restrict the smoothening of the patch and to allow a proper soaking. IPM proved to be a particularly successful solvent.

### Example 2

### Testing of Further Solvents

Objective

It should be analysed if Polidocanol is as good as IPM for application of rasagiline.

Description

The strategy of applying a rasagiline solution onto a placebo patch (particularly by dropping onto the patch) yielded good results. The solvent IPM has the disadvantage of being a penetration enhancer. Alternative solvents should be identified that lack a penetration enhancing effect.

Methods

2.1 Application of ca. 20 mg Polidocanol onto the placebo patches RAS021TS_D001, RAS022TS_D001, or RAS023TS_D001.

Result: Polidocanol permeates the patch.

2.2 Solubility of Rasagiline in Polidocanol

Mixing of 48.5 mg rasagiline and 147.0 mg polidocanol

Result: rasagiline is completely soluble.

3.3 Preparation of Placebo Laminates Using Plastoid B

Siehe manufacturing protocol for RAS032TS_D001, RAS033TS_D001 or RAS034TS_D001
From the prepared placebo laminates TDS patches with an area of 10 cm² were punched out
ca. 20 mg of a rasagiline polidocanol (1:3) solution are applied, target concentration: c(rasagiline)=5 mg/TDS
the patch is allowed to stand for 60 min, during this time the solution permeates the patch
thereafter the Patch is laminated with a back up layer (FL2000 Liner)
after the residence time, TDS with an area of 1.5 cm² are punched out for in vitro analysis of skin permeation

Aspect of the patches:
RAS032TS_D001 (20% Plastoid B): adhesive, slightly slumped, slightly fatty TDS without wave-like structure
RAS033TS_D001 (40% Plastoid B): sparsely adhesive, slightly slumped, slightly fatty TDS without wave-like structure
RAS034TS_D001 (60% Plastoid B): non adhesive to sparsely adhesive, slightly slumped, slightly fatty TDS without wave-like structure

Conclusion

Based on the results polidocanol is a suitable solvent for rasagiline. Furthermore the solvent completely permeates the patch, which constitutes an ideal requirement for the further development. It is known that polidocanol is hardly released from an acrylate matrix. Therefore it can be expected that no or only a limited co-diffusion of rasagiline and polidocanol will take place.

The prototypes RAS033TS_D001 and RAS034TS_D001 were further analysed in a skin permeation experiment and revealed an excellent cumulative skin permeation rate that was even higher as the permeation rate observed for the Plastoid-containing patches (see FIG. 5). Due to the fact that polidocanol exhibits pharmacological effects, additional experiments were undertaken to identify structural similar solvents of high molecular weight.

### Example 3

### Preparation and Testing of a Two-Layer Prototype

Background

In pre-tests the Plastoid B was initially mixed with the adhesive matrix. This resulted in patches with a very little adhesive force. In order to overcome this problem the strategy of a multiple layer prototype was tested build up from an adhesive layer and a plastoid layer.

Methods

The plastoid layer is created using a 2% Miglyol solution. Therefore 0.1 g Plastoid B (WE8656) were solubilised in Miglyol 812 (WE8884) ad 5.0 g.

As an adhesive layer DuroTak 4287 (WE7984, solid content ˜38%) is used. The adhesive is streaked onto FL2000 in a way that the dry area-weight equates ca. 60 g/m². The layers are dried at room temperature for 10 min, followed by 30 min at 70° C. A web was streaked and the resulting laminate bisected.

For the application of the Plastoid two variants were tested:
(1) The plastoid-layer is directly coated onto the dried adhesive streak. The coating weight accounts for 40 g/m². The laminate was dried for 10 to 60 min at RT. The resulting laminate was coated with Hostaphan RN15 (WE6776).
(2) The plastoid layer is streaked onto Hostaphan RN15. The coating weight accounts for 40 g/m². The laminate was dried for 10 min to become touch dry and further coated with an adhesive layer.
Processing and laminate aspect were evaluated.

Results

Experiment 3.1: Plastoid does not solubilize as 2% solution in miglyol. The plastoid remains as unsolubilised crystals. In further experiments (see exp.; 3.2 and 3.3) the solution was warmed with caution while stirring to a temperature of ca. 50° C. The plastoid dissolves and remains solubilised. The resulting solution exhibits a very low viscosity.

Experiment 3.2: Herein Eudragit E was used for a multi-layer TDS. Eudragit E and Miglyol were weighed in a ratio of 1:1 in an aluminum dish and mixed with a Pasteur pipet. The mixture which was very dry in the beginning liquefied during the time to result in a viscous clear mass (viscosity similar to viscous honey).

Experiment 3.3: Plastoid B and Miglyol were weighed in a ratio of 1:1 in an aluminum dish and mixed which resulted in a crystalline mass. This was heated in a heating oven at 50° C. yielding a clear “clump”. After addition of Miglyol to obtain a ration of 1:2 and further heating at 50° C., a viscous clear mass similar to exp. 3.2. was obtained (viscosity similar to viscous honey).

Experiment 3.4: A 12% Plastoid B solution as prepared. This solution was stirred at ca. 50° C. until the Plastoid was completely dissolved. After 3 hours the solution was clear, however, undissolved plastoid particles could be seen at the border of the glass. The viscosity of the resulting solution is too low to be streaked (even after cooling). After two days of holding time the solution becomes more viscous.

Experiment 3.5: A 25% Plastoid B solution as prepared. This solution was stirred at ca. 50 to 60° C. until the Plastoid was completely dissolved. After 2.5 hours the solution was clear, no plastoid particles could be observed. Accordingly neutral oil was added. The resulting 20% solution still exhibits a high viscosity. It was found that a solution of 16.67% possesses an acceptable viscosity. This solution was further processed in experiment 3.6.

Experiment 3.6: A placebo laminate was prepared by streaking Durotak 4287 with a dry area weight of 60 g/m².

Web No 1. R.III 200 μm dry area weight of 69.177 g/m²
(1) Plastoid solution was coated onto a dried adhesive layer with a thickness of 110 μm, dried at RT for 60 min. Total area weight=103.115 g/m² (rel. std. dev. 9.5%); Aspect: very soft matrix—in experiment 3.7 it should be tried to solidify the matrix by increasing the plastoid concentration
(2) Not possible, since the plastoid solution is smearing during the coating process

Experiment 3.7:

The batch RAS035TS_D001 was prepared. The Plastoid Bconcentration in the final laminate should equate 20%.
Area weight adhesive matrix=70 g/m²
Total area weight: 120 g/m² (rel. std. dev. 13.8%)
Theoretical area weight neutral oil-Plastoid B: 50 g/m²
Aspect: clear an homogenous laminate, soft matrix

Experiment 3.8: rasagiline solubility in a Plastoid Bneutral oil solution
50 mg rasagiline WE 8596 in 300 mg Plastoid B-solution from exp. 3.5
Rasagiline is dissolved after 10 min, resulting in a clear, slightly yellow solution

Experiment 3.9: Placebo-TDS with Plastoid B-neutral Oil Solution

The batch RAS036TS_D001 was prepared by using three different concentrations of Plastoid B (12.5%, 22.5% or 32.5%). The coating of the neutral oil-Plastoid Bsolution is performed according to the manufacturer's protocol:

The FL2000 layer is aspirated with the non-siliconized side onto the coating plate. The adhesive laminate pieces are attached with an adhesive tape on the liner, so that centrally and at the edges an open area is produced allowing a free guidance of the scraper. The scraper adjustment is calculated based on the foil thickness, area weight of the adhesive matrix, plus the targeted area weight of the Plastoid Bneutral oil solution Targeted area weight of the adhesive matrix 80 g/m²

Targeted total area weight (Placebo) 95 g/m²

12.5% Plastoid B
Area weight of the adhesive matrix: 78.577 g/m²
Total area weight: 94.128 g/m² (rel. std dev 1.14%)
Theoretical area weight of the plastoid neutral oil solution: 15.55 g/m²
Aspect: clear homogenous laminate, detach from the skin like chewing gum

22.5% Plastoid B
Area weight of the adhesive matrix: 76.143 g/m²
Total area weight: 89.092 g/m² (rel. std. dev. 2.02%)
Theoretical area weight of the plastoid neutral oil solution: 12.95 g/m²
Aspect: clear homogenous laminate

32.5% Plastoid B
Area weight of the adhesive matrix: 80.334 g/m²
Total area weight: 97.670 g/m² (rel. std dev. 5.88%)
Theoretical area weight of the Plastoid Bneutral oil solution: 17.34 g/m²
Aspect: clear homogenous laminate

Experiment 3.10: Rasagiline TDS with a Plastoid B-Neutral Oil Solution

Since the laminates produced in exp. 3.9 possess good adhesive properties they were produced as API containing patches of the following batches: RAS037TS_D001 (32.5% Plastoid B); RAS038TS_D001 (22.5% Plastoid B)

Preparation protocol see example 6

Targeted area weight of the adhesive matrix 80 g/m²

Targeted total area weight (Placebo) 100 g/m²

RAS037TS_D001
Area weight of the adhesive matrix: 84.559 g/m²
Total area weight: 101.243 g/m² (rel. Std dev 12.40%)
Theoretical area weight of the Plastoid Bneutral oil solution: 16.684 g/m²
Aspect: clear homogenous laminate

RAS038TS_D001
Area weight of the adhesive matrix: 82,452 g/m²
Total area weight: 101.463 g/m² (rel. Std dev 4.08%)
Theoretical area weight of the Plastoid Bneutral oil solution: 19.011 g/m²
Aspect: clear homogenous laminate

From each batch 6 patches were analysed in an in vitro skin permeation assay. The batch RAS022TS_D001 was used as reference.

Conclusion

The rasagiline TDS resulted at both Plastoid Bconcentrations in a good aspect.

### Example 4

### Testing of Macrogol Ether and -Ester as Solvent

Background

Previous experiments showed that polidocanol is a very good solvent for rasagiline. However, the polidocanol possesses a pharmaceutical effect (antipruritic) which could pose regulatory problems. Therefore polidocanol analogues with longer chains and lacking pharmacological effects were analysed. Furthermore also macrogol esters were tested. Since these substances are solid compounds it was tried to melt them for further processing.

Method

It was tried to liquefy the compounds Brij S20, Brij CS20, Crodet S40 and Cithrol 10 MS at a temperature of 40° C.

Ca. 0.5 g were weighed in a beaker glass and heated on a heating plate up to 40° C. while the melting characteristics of the compounds were observed.

Results

Ca. 0.5 g were weighed in a beaker glass and heated on a heating plate while stirring. The heating plate had a temperature of ca. 65-70° C., resulting in a melting temperature of ca. 40-50° C. within the glass.

| Solid compound | Appearance at ca. 40° C. | Comment |
|---|---|---|
| Brij S20 (Pellets) | Melts quickly: ca. 1 min | |
| Brij CS20 (Pellets) | Melts within ca. 3 min | |
| Crodet S40 (Pellets) | Melts within 5 min | Platte was not so |
| | | hot anymore: ca. |
| | | 55° C. |
| Cithrol 10 MS | Melts quickly: ca. 1 min | |
| (compound is solid, | | |
| small particles were | | |
| scrapped from the | | |
| block) | | |

Within 1-2 minutes all compounds become solid again.

Conclusion

All solid compounds can be melted at a low temperature therefore permitting a processing. However, the experiment shows also that the molten compounds will quickly become solid again when the temperature is lowered.

### Example 5

### Placebo and Rasagiline TDS with Macrogol Derivates

Goal

The solubility of the rasagiline in the macrogol derivatives should be analysed and acrylate patches should be prepared using a placebo or an API-containing solution.

Methods

The above listed substances were melted at 40° C. From each molten substance 20 mg were dropped onto a placebo acrylate patch and dispersed. As placebo patch rectangular 10 cm² die-cut sheets from the laminate RAS022TS_D001 were used. The handling and the soaking characteristics were documented. Macrogol derivates with good processability were analysed as follows:

210 mg of the macrogol derivate was molten and 70 mg rasagiline were added. The salvation characteristics of the rasagiline was observed and documented. The solvation process has to be performed under stirring.

API containing patches were produced using the macrogol derivates with good solubility of rasagiline. The handling and the soaking characteristics were documented.

Die resulting patches were analysed for skin permeation, for content/purity or presence of crystals.

Results

| Macrogol derivates | comment/description |
|---|---|
| Cithrol 10 MS | The molten macrogol derivative solidifies |
| | during the application on the TDS. It can't be |
| | dissolved evenly.. |
| Brij S20 | Forceps were warmed for application. The |
| | application is more practical, also here the |
| | macrogol solidifies quickly. |

Both samples were cautiously warmed on a heating plate whereby the
magrogol melts again and the derivative can be dispersed. Both TDS
samples were kept for ca. 45 minutes on the heating plate at this
temperature. The macrogol derivatives contract and generate drops but
do not permeate the adhesive matrix. After cooling of the patches the
macrogol derivates solidify again creating a wax-like sheet on the TDS.

| | |
|---|---|
| Brij CS20 | Forceps were warmed for application. The |
| | application is more practical, also here the |
| | macrogol solidifies quickly. |
| Crodet S40 | Forceps were warmed for application. The |
| | application is more practical, also here the |
| | macrogol solidifies quickly. |

Both samples were cautiously warmed on a heating plate whereby the
magrogol melts again and the derivative can be dispersed. Both TDS
samples were kept for ca. 30 minutes on the heating plate at this
temperature. The macrogol derivatives contract and generate drops but
do not permeate the adhesive matrix. After cooling of the patches the
macrogol derivates solidify again creating a wax-like sheet on the TDS.

For the following experiments the pipets and the placebo TDS were pre-warmed in a drying oven. The TDS batch RAS022TS (round, area of 10 cm²) was used.

Experiment 5.1:

70 mg rasagiline was weighed into a small screw cap glass. Cithrol 10MS was melted in a drying oven at 50° C. and 210 mg molten Cithrol was added with the pre-warmed pipet to the rasagiline.

Observation:

During the addition the rasagiline melts. After cooling the mixture solidifies quickly.

The mixture of rasagiline and Cithrol is melted for 2 minutes in the drying oven and homogenized subsequently for ca. 1 minute. 20 mg of this mixture is applied on to a pre-warmed placebo and dispersed using a Pasteur pipet. The TDS is covered and cooled. The rasagiline-macrogol derivate mixture solidifies unevenly and criss-crosses on the TDS.

Experiment 5.2:

70 mg rasagiline was weighed into a small screw cap glass. Brij S20 was melted in a drying oven at 50° C. and 210 mg molten Brij S20 was added with the pre-warmed pipet to the rasagiline.

Result:

The added Brij S20 solidifies more quickly, resulting in a “wax-like drop”. The mixture is melted in a drying oven, whereby the rasagiline is molten within 2 minutes and the Brij within further 7 minutes. The molten mixture is homogenized subsequently for ca. 1 minute.

20 mg of this mixture is applied on to a pre-warmed placebo and dispersed using a Pasteur pipet whereby the mixture solidifies very quickly. The mixture is melted again in the drying oven and dispersed again. The TDS is covered and cooled. The rasagiline-macrogol derivate mixture solidifies quickly but disperses more evenly and was coated with a adhesive silicone matrix.

Experiment 5.3:

70 mg rasagiline was weighed into a small screw cap glass. CrodetS40 was melted in a drying oven at 55° C. and 210 mg molten CrodetS40 was added with the pre-warmed pipet to the rasagiline.

Result:

The added CrodetS40 solidifies very quickly, resulting in a “wax-like drop”. The mixture is melted in a drying oven, whereby the rasagiline is molten within 2 minutes and the Crodet within further 7 minutes. The molten mixture is homogenized subsequently for ca. 1 minute.

20 mg of this mixture is applied on to a pre-warmed placebo and dispersed using a Pasteur pipet whereby the mixture solidifies very quickly. As a result it was not possible to disperse the mixture evenly on the TDS. The mixture is melted again in the drying oven and dispersed again. The TDS is covered and cooled. The rasagiline-macrogol derivate mixture solidifies quickly but disperses more evenly and was coated with an adhesive silicone matrix.

As a reference for the skin permeation experiments, a “fresh” TDS using IPM as solvent was prepared. Therefore 70 mg rasagiline were solubilised in IPM. 20 mg of this solution were applied on a 10 cm² acrylate patch and dispersed evenly. (In analogy to example 1).

Conclusion

It is possible to produce TDS patches by slight warming treatment. BrijS20 and Crodet are particularly suited as solvents for rasagiline. Since the macrogol derivates form a wax-like sheet, they are coated with a thin silicone layer (ca. 20 G/m²) as “skin streak”. The samples with the following batch numbers wee tested for skin permeation:
RAS022TS_D001_BrijS20 (content rasagiline: 0.63 mg/1.5 cm²)
RAS022TS_D001_Crodet (content rasagiline: 0.68 mg/1.5 cm²)

As reference a freshly prepared patch of the same batch without silicone layer with IPM as solvent was used:
RAS022TS_D001_IPM (content rasagiline: 0.84 mg/1.5 cm²)

Analysis of the patches RAS022TD containing Brij or Crodet regarding skin permeability revealed a high cumulative permeation rate for both patches which is similar to the Plastoid containing reference patch (see FIG. 4).

### Example 6

### Preparation of a Rasagiline Multilayer TDS

Goal

Based on a solution of rasagiline in neutral oil and Plastoid B a multi-layer TDS should be prepared.

Methods

The following substances were mixed to generate Mixture A:
0.35 g rasagiline base
0.175 g Plastoid B
0.875 g Neutral oil

The following substances were mixed to generate Mixture B:
9.035 g Durotak 87-4287
2.1 g Plastoid B
7.465 g ethyl acetate

The preparation of the patch includes the following steps: The adhesive laminate pieces are attached with an adhesive tape on the liner, so that centrally and at the edges an open area is produced allowing a free guidance of the scraper.
The liner is aspirated onto the coating plate (mixture B)
The adhesive laminate pieces are attached with an adhesive tape on the liner, so that centrally and at the edges an open area is produced allowing a free guidance of the scraper.
Scraper adjustment: 210 μm
Rasagiline solution (Mixture A) is applied on the adhesive layer
Solution permeates for 60 minutes in the dark
Laminate is coated with a back up layer (Trespaphan)
Samples are punched out for skin permeation experiments

Thus, the patches according to the present invention having an area of about 1 to 50 cm² were able to transdermally provide a therapeutic dose needed for up to 7 days treatment, and the penetration rate of rasagiline could be controlled by using multilayer patches. Since the raw materials for preparing the patches are widely and readily available, and rasagiline could be transferred through the whole surface of the substrate for transdermal absorption, the patches of the present invention could be prepared simply with widely available raw materials, have smooth release profiles and a prolonged period for controlled release, and may be more effectively absorbed through skin in comparison with the system as described in WO 2007/101400.

1. A method for preparing a transdermal patch comprising a substrate layer, the method comprising the steps of:
a) contacting an active pharmaceutical ingredient with a retaining means to provide a composition; and
b) applying the composition obtained in step (a) to a carrier material to form a substrate layer of the transdermal patch;
wherein the retaining means remains within the final transdermal patch.

2. The method according to claim 1, wherein the carrier material is a dried layer of polymer matrix, preferably a dried layer of adhesive polymer matrix.

3. The method according to claim 1, wherein the active pharmaceutical ingredient has a chemical structure according to one of the following formulas: 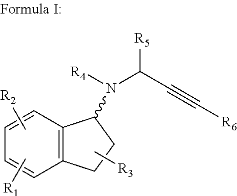
wherein
R₁ is hydrogen, halogen, alkyl, alkoxy, acyl, acyloxy, aryl, aralkyl, hydroxy, carboxy, amine, alkylamine, dialkylamine, nitro, or —OC(O)NR₁₂R₁₃, and may be substituted by one or more substituents selected from alkyl, halogen, hydroxy, carboxy, amine, alkylamine, dialkylamine;
R₂, R₃, R₄, R₅, R₆, R₁₂ and R₁₃ independent from each other are hydrogen, halogen or alkyl;
and wherein one or more, preferably one or two, of the carbon atoms in the formula including the substituents may be replaced by a heteroatom such as nitrogen, oxygen or sulfur; and 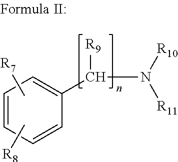
wherein
R₇ is hydrogen, halogen, alkyl, alkoxy, acyl, acyloxy, aryl, aralkyl, hydroxy, carboxy, amine, alkylamine, dialkylamine, nitro, or —OC(O)NR₁₄R₁₅, and may be substituted by one or more substituents selected from alkyl, halogen, hydroxy, carboxy, amine, alkylamine, dialkylamine;
R₈, R₉, R₁₀, R₁₄ and R₁₅ independent from each other are hydrogen, halogen or alkyl, wherein if more than one R₉ is present, these R₉ groups may also be different from each other;
R₁₁ is hydrogen, halogen or alkyl optionally substituted by halogen or hydroxy;
n is an integer from 1 to 4, preferably 1 or 2;
and wherein one or more, preferably one or two, of the carbon atoms in the formula including the substituents may be replaced by a heteroatom such as nitrogen, oxygen or sulfur.

4. The method according to claim 1, wherein the active pharmaceutical ingredient is a volatile substance and preferably is rasagiline, selegiline, rivastigmine or ladostigil, or a derivative thereof, preferably in the form of the free base.

5. The method according claim 1, wherein the retaining means is a liquid, preferably a pharmaceutically acceptable low volatile solvent, more preferably selected from the group consisting of synthetic or natural oils, isopropyl myristate, polyethoxylated fatty acids and polyethoxylated fatty alcohols such as polidocanol, Brij-, Crodet-, Myrj-, Atlas-types, and mixtures thereof, and the active pharmaceutical ingredient is contacted with the retaining means in step (a) to form a solution, suspension or emulsion.

6. The method according to claim 5, wherein a soaking additive is further added to the either the carrier material or the drug solution.

7. The method according to claim 1, wherein the retaining means is a plasticizer and the carrier material is a mixture of an adhesive polymer and a non-adhesive polymer.

8. The method according to claim 1, wherein the retaining means is solid, preferably selected from the group consisting of higher molecular weight polyethoxylated fatty acids and polyethoxylated fatty alcohols, PVP, PEO, PVA, PVPVA, cellulose and derivatives, starch and derivatives and their blends.

9. The method according to claim 8, wherein the active pharmaceutical ingredient is
(i) contacted with the retaining means in step (a) in a molten state, mixed, solidified and ground to form a solid mixture, or
(ii) attached to the surface of the retaining means to form a coated or impregnated material.

10. The method according to claim 1, wherein the carrier material comprises at least one type of the following polymer materials: polyacrylates and derivatives thereof, silicone polymers and derivatives thereof, polyisobutylene and derivatives thereof, ethylene-vinyl acetate copolymers and derivatives thereof, styrene-block-co-polymers and derivatives thereof, POX and derivatives thereof, polyurethanes and derivatives thereof, polyolefines and derivatives thereof, polyesters and derivatives thereof, and polyacrylic acids and derivatives thereof.

11. The method according to claim 1, wherein the carrier material is an adhesive, preferably an adhesive acrylate, polyurethane, polyisobutylene or styrene-block-co-polymer.

12. The method according to claim 1, wherein the carrier material contains a soaking additive, preferably Plastoid B or Eudragit.

13. The method according to claim 1, wherein the carrier material contains a non-adhesive, preferably a non-adhesive acrylate or a fleece material, and an adhesive layer is further introduced to form the transdermal patch.

14. The method according to claim 1, wherein the transdermal patch further comprises a backing layer, a protective layer and/or a release controlling layer such as a membrane having a defined pore size.

15. The method according to claim 1, wherein the transdermal patch comprises an adhesive layer for contacting the skin, which is adjacent to the substrate layer or adjacent to an optionally present release controlling layer.

16. The method according to claim 1, wherein the method comprises the following steps:
(i) a) adsorbing the active pharmaceutical ingredient onto the solid retaining means to form a composition, preferably by melting the active pharmaceutical ingredient in presence of the retaining means and then solidifying;
b) grinding the composition;
c) mixing the ground composition with molten carrier material or with a solution comprising the carrier material;
d) applying the dispersion or solution onto a backing layer to form a film; and
e) cooling or drying the film to form a substrate layer;
or
(ii) a) forming an adhesive matrix layer comprising the carrier material;
b) mixing the active pharmaceutical ingredient with the liquid retaining means to form a solution or suspension;
c) applying the solution or suspension to the matrix layer to form a substrate layer, preferably by soaking, printing or casting;
or
(iii) a) forming an adhesive matrix layer comprising the carrier material;
b) mixing the active pharmaceutical ingredient with the liquid retaining means and a non-adhesive carrier material to form a viscous solution or suspension;
c) applying the solution or suspension to the matrix layer to form a substrate layer, preferably by spreading; and
d) optionally applying another adhesive layer;
or
(iv) a) forming an adhesive matrix layer comprising the carrier material;
b) attaching the solid retaining means, preferably a fleece material, to the adhesive matrix layer;
c) applying the active pharmaceutical ingredient to the retaining means, for example by impregnating, either before or after the retaining means is attached to the adhesive matrix layer; and
d) applying another adhesive layer to the other site of the retaining means;
or
(v) a) forming an adhesive matrix layer comprising the carrier material;
b) adsorbing the active pharmaceutical ingredient onto the solid retaining means to form a composition, preferably by melting the active pharmaceutical ingredient in presence of the retaining means and then solidifying;
c) grinding the composition;
d) applying the ground composition to the adhesive matrix layer to form a substrate layer; and
e) optionally applying another adhesive layer;
or
(vi) a) forming an adhesive matrix layer comprising the carrier material, e.g. by coating or laminating;
b) applying the active pharmaceutical ingredient to the solid retaining means to form a composition by mixing the active pharmaceutical ingredient and the retaining means in the presence of a highly volatile solvent;
c) applying the composition to the adhesive matrix layer to form a substrate layer;
d) drying the substrate layer; and
e) optionally applying another adhesive layer.

17. A transdermal patch comprising a backing layer and a substrate layer comprising a volatile active pharmaceutical ingredient, a carrier material and a retaining agent.

18. The transdermal patch according to claim 17, which is obtainable by a method according to claim 1.

19. The transdermal patch according to claim 17, wherein the active pharmaceutical ingredient has a chemical structure according to one of the following formulas: 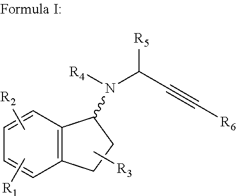
wherein
R₁ is hydrogen, halogen, alkyl, alkoxy, acyl, acyloxy, aryl, aralkyl, hydroxy, carboxy, amine, alkylamine, dialkylamine, nitro, or —OC(O)NR₁₂R₁₃, and may be substituted by one or more substituents selected from alkyl, halogen, hydroxy, carboxy, amine, alkylamine, dialkylamine;
R₂, R₃, R₄, R₅, R₆, R₁₂ and R₁₃ independent from each other are hydrogen, halogen or alkyl;
and wherein one or more, preferably one or two, of the carbon atoms in the formula including the substituents may be replaced by a heteroatom such as nitrogen, oxygen or sulfur; and 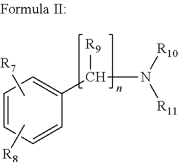
wherein
R₇ is hydrogen, halogen, alkyl, alkoxy, acyl, acyloxy, aryl, aralkyl, hydroxy, carboxy, amine, alkylamine, dialkylamine, nitro, or —OC(O)NR₁₄R₁₅, and may be substituted by one or more substituents selected from alkyl, halogen, hydroxy, carboxy, amine, alkylamine, dialkylamine;
R₈, R₉, R₁₀, R₁₄ and R₁₅ independent from each other are hydrogen, halogen or alkyl, wherein if more than one R₉ is present, these R₉ groups may also be different from each other;
R₁₁ is hydrogen, halogen or alkyl optionally substituted by halogen or hydroxy;
n is an integer from 1 to 4, preferably 1 or 2;
and wherein one or more, preferably one or two, of the carbon atoms in the formula including the substituents may be replaced by a heteroatom such as nitrogen, oxygen or sulfur.

20. The transdermal patch according to claim 17, wherein the active pharmaceutical ingredient is a volatile substance and preferably is rasagiline, selegiline, rivastigmine or ladostigil, or a derivative thereof, preferably in the form of the free base.

21. The transdermal patch according to claim 17, wherein the retaining means is a liquid, preferably a pharmaceutically acceptable low volatile solvent, more preferably selected from the group consisting of natural or synthetic oils, fatty acid esters, such as isopropyl myristate, polyethoxylated fatty acids and polyethoxylated fatty alcohols such as polidocanol, and mixtures thereof.

22. The transdermal patch according to claim 17, wherein the retaining means is a plasticizer and the carrier material is a mixture of an adhesive polymer and a non-adhesive polymer.

23. The transdermal patch according to claim 17, wherein the retaining means is a solid, preferably a pharmaceutically acceptable solid, more preferably selected from the group consisting of higher molecular weight polyethoxylated fatty acids and polyethoxylated fatty alcohols, PVP, PVA, PVPVA, PEO, cellulose and derivatives, starch and derivatives or their blends.

24. The transdermal patch according to claim 17, wherein the transdermal patch contains at least 0.1% w/w of the retaining means.

25. The transdermal patch according to claim 17, wherein the carrier material comprises at least one type of the following polymer materials: polyacrylates and derivatives thereof, silicone polymers and derivatives thereof, polyisobutylene and derivatives thereof, ethylene-vinyl acetate copolymers and derivatives thereof, Styrene-block-co-polymers and derivatives thereof, PDX and derivatives thereof, polyurethanes and derivatives thereof, polyolefines and derivatives thereof, polyesters and derivatives thereof. and polyacrylic acids and derivatives thereof.

26. The transdermal patch according to claim 17, wherein the carrier material is an adhesive, preferably an adhesive acrylate, polyurethane, polyisobutylene or styrene-block-co-polymer.

27. The transdermal patch according to claim 17, wherein the carrier material contains a soaking additive, preferably Plastoid B or Eudragit.

28. The transdermal patch according to claim 17, wherein the carrier material contains a non-adhesive, preferably a non-adhesive acrylate or a fleece material, and the transdermal patch further comprises an adhesive layer.

29. The transdermal patch according to claim 17, further comprising a backing layer, a protective layer and/or a release controlling layer such as a membrane having a defined pore size.

30. The transdermal patch according to claim 17, wherein the substrate layer further comprises a non-adhesive polymer, preferably a non-adhesive acrylate, and/or a soaking additive.

31. The transdermal patch according to claim 17 for use in medicine.

32. The transdermal patch according to claim 31, wherein the active pharmaceutical ingredient is rasagiline or a derivative thereof, preferably in the form of the free base, for treatment or prophylaxis of a nervous system disease, preferably a nervous system disease selected from the group consisting of Parkinson's disease, Alzheimer's disease, depression, hyperactive child syndrome, restless leg syndrome, multiple sclerosis and abstinence syndrome.
